(19) United States
(12) Patent Application Publication
(10) Pub. No.: US 2007/0049918 A1
(43) Pub. Date: Mar. 1, 2007

(54) Microwave device for vascular ablation

(76) Inventors: Daniel Warren van der Weide, Madison, WI (US); Fred T. Lee JR., Madison, WI (US); Christopher L. Brace, Middleton, WI (US); Paul F. Laeseke, Madison, WI (US)

Correspondence Address: Patula & Associates, P.C., 14th Floor, 116 S. Michigan Ave., Chicago, IL, 60603, US

(21) Appl. No.: 11/509,123

(22) Filed: Aug. 24, 2006

Related U.S. Application Data
(63) Continuation-in-part of application No. 11/237,136, filed on Sep. 28, 2005.
(63) Continuation-in-part of application No. 11/237,430, filed on Sep. 28, 2005.
(63) Continuation-in-part of application No. 11/236,985, filed on Sep. 28, 2005.
(63) Continuation-in-part of application No. 11/440,331, filed on May 24, 2006.
(63) Continuation-in-part of application No. 11/452,637, filed on Jun. 14, 2006.
(63) Continuation-in-part of application No. 11/502,783, filed on Aug. 11, 2006.
(60) Provisional application No. 60/710,815, filed on Aug. 24, 2005.

(51) Int. Cl. A61B 18/18

(52) U.S. Cl. 606/33

(57) ABSTRACT

A method and system delivers microwave energy to a vessel, such as a vein for the treatment of varicose veins, in a controllable heating pattern and to provide relatively fast heating and ablation of the vessel. The method and system comprises a microwave delivery device for heating the vessel, and a microwave power source for supplying microwave power to the delivery device. The method and system may also include a cooling system, a temperature monitoring, feedback and control system, an ultrasound or other imaging device, and/or a device for assuring generally uniform energy delivery in the vessel.

### CLAIM OF PRIORITY

This application is a Continuation-In-Part of co-pending U.S. Non-Provisional Patent Applications entitled “Segmented Catheter for Tissue Ablation” filed Sep. 28, 2005 and assigned U.S. application Ser. No. 11/237,136; “Cannula Cooling and Positioning Device” filed Sep. 28, 2005 and assigned U.S. application Ser. No. 11/237,430; “Air-Core Microwave Ablation Antennas” filed Sep. 28, 2005 and assigned U.S. application Ser. No. 11/236,985; “Microwave Surgical Device” filed May 24, 2006 and assigned U.S. application Ser. No. 11/440,331; “Microwave Tissue Resection Tool” filed Jun. 14, 2006 and assigned U.S. application Ser. No. 11/452,637; and “Intralumenal Microwave Device” filed Aug. 11, 2006 and assigned U.S. application Ser. No. 11/502,783; the entire disclosures of each and all of these applications are hereby herein incorporated by reference.

This application further claims priority, where applicable, to U.S. Provisional Patent Applications entitled “Segmented Catheter for Tissue Ablation” filed May 10, 2005 and assigned U.S. Application Ser. No. 60/679,722; “Microwave Surgical Device” filed May 24, 2005 and assigned U.S. Application Ser. No. 60/684,065; “Microwave Tissue Resection Tool” filed Jun. 14, 2005 and assigned U.S. Application Ser. No. 60/690,370; “Cannula Cooling and Positioning Device” filed Jul. 25, 2005 and assigned U.S. Application Ser. No. 60/702,393; “Intralumenal Microwave Device” filed Aug. 12, 2005 and assigned U.S. Application Ser. No. 60/707,797; “Air-Core Microwave Ablation Antennas” filed Aug. 22, 2005 and assigned U.S. Application Ser. No. 60/710,276; and “Microwave Device for Vascular Ablation” filed Aug. 24, 2005 and assigned U.S. Application Ser. No. 60/710,815; the entire disclosures of each and all of these applications are hereby herein incorporated by reference.

### CROSS-REFERENCE TO RELATED APPLICATIONS

This application is related to co-pending U.S. Non-Provisional patent applications entitled “Triaxial Antenna for Microwave Tissue Ablation” filed Apr. 29, 2004 and assigned U.S. application Ser. No. 10/834,802; “Segmented Catheter for Tissue Ablation” filed Sep. 28, 2005 and assigned U.S. application Ser. No. 11/237,136; “Cannula Cooling and Positioning Device” filed Sep. 28, 2005 and assigned U.S. application Ser. No. 11/237,430; “Air-Core Microwave Ablation Antennas” filed Sep. 28, 2005 and assigned U.S. application Ser. No. 11/236,985; “Microwave Surgical Device” filed May 24, 2006 and assigned U.S. application Ser. No. 11/440,331; “Microwave Tissue Resection Tool” filed Jun. 14, 2006 and assigned U.S. application Ser. No. 11/452,637; and “Intralumenal Microwave Device” filed Aug. 11, 2006 and assigned U.S. application Ser. No. 11/502,783; and to U.S. Provisional Patent Applications entitled “Segmented Catheter for Tissue Ablation” filed May 10, 2005 and assigned U.S. Application Ser. No. 60/679,722; “Microwave Surgical Device” filed May 24, 2005 and assigned U.S. Application Ser. No. 60/684,065; “Microwave Tissue Resection Tool” filed Jun. 14, 2005 and assigned U.S. Application Ser. No. 60/690,370; “Cannula Cooling and Positioning Device” filed Jul. 25, 2005 and assigned U.S. Application Ser. No. 60/702,393; “Intralumenal Microwave Device” filed Aug. 12, 2005 and assigned U.S. Application Ser. No. 60/707,797; “Air-Core Microwave Ablation Antennas” filed Aug. 22, 2005 and assigned U.S. Application Ser. No. 60/710,276; and “Microwave Device for Vascular Ablation” filed Aug. 24, 2005 and assigned U.S. Application Ser. No. 60/710,815; the entire disclosures of each and all of these applications are hereby herein incorporated by reference.

### FIELD OF THE INVENTION

The present disclosure relates generally to the field of vascular ablation or venous ablation, and the delivery of microwave energy to treat vascular pathologies. Specifically, the present disclosure relates to a method and system for the controlled delivery of microwave power to a vessel wall, and in particular a vein, to treat vascular pathologies such as varicose veins, port wine stains, arterio-venous malformations, pseudoaneurysms, aneurysms, spider angiomas, hemangiomas, venous leakage as a cause for impotence, and other vascular pathologies.

### BACKGROUND

Varicose veins are a common medical condition that affect up to 60% of all Americans, and represent a significant health and cosmetic problem. Symptomatically, dilated varicose veins (usually the greater saphenous vein) can cause pain, cramping, itching, swelling, skin changes, venous stasis ulcers, and aching. The traditional therapy for treatment of varicose veins has been surgical removal (vein stripping), but currently less invasive treatments are becoming more common. Sclerotherapy (injection of a caustic substance to scar down the vein), laser and radiofrequency closure techniques, and minimally invasive surgery are becoming more popular. Energy delivery treatments (laser, radiofrequency, etc.) are promising because of their relatively low technical difficulty and good accuracy.

Limitations of the above techniques center on the means by which the vein in treated. Surgical techniques can be technically challenging and more invasive than energy delivery techniques or sclerotherapy. Sclerotherapy is limited in the accuracy by which substances may be administered. Laser techniques can cause the vein to become extremely hot, which increases the probability of burns to the skin and subcutaneous tissues as well as perforation of the vein. Radiofrequency techniques are relatively slow to heat, require ground pads to be placed on the patient and are not precise.

Accordingly, there is a need for a new and improved method and system to treat vascular pathologies such as varicose veins, which overcomes the above identified disadvantages and limitations of current vascular pathology and varicose vein treatment methods. The present disclosure fulfills this need.

### SUMMARY

The present disclosure relates to a method and system for vascular ablation using microwave energy to provide a very controllable heating pattern and to provide relatively fast heating, much faster for example than radiofrequency energy heating. The method and system delivers microwave (e.g. approximately 300 MHz and higher frequencies) power to a vessel wall, in particular for the treatment of vascular pathologies such as varicose veins.

The vascular ablation system generally comprises a microwave delivery device for heating the vessel wall, and a microwave power source for supplying microwave power to the delivery device. The vascular ablation system also preferably may include a cooling system, a temperature monitoring, feedback and control system, an ultrasound or other imaging device, and/or a device for assuring generally uniform energy delivery in the vein.

In a first embodiment, the microwave delivery device comprises a very thin microwave antenna that can be placed into the lumen of the vein. Focused microwave energy from an extracorporeal microwave power source would then be directed at this antenna transcutaneously to cause heating of the vessel wall and closure of the vein. Ferrite (or similar material) may be incorporated into the antenna wire to increase the heating effect of the external microwave field. Advantages of this approach include: (1) the intraluminal antenna could be very thin and minimally traumatic when placed inside the vein, (2) external heating could be primarily directed at the visible vessels on the leg surface, and (3) the external approach increases certainty of location of heat delivery, thus minimizing technical difficulty and reheating of already treated veins.

In a second embodiment, the microwave delivery device comprises a microwave antenna built into an endoluminal catheter that is specifically tuned to the impedance of the vessel wall. This tuning reduces reflected power, allowing the catheter to be very thin, reducing the trauma of antenna placement into the vein. The catheter could be a triaxial microwave catheter or other microwave antenna including center-fed dipole, dual-feed slot, segmented, or other microwave antennas. In this embodiment, the microwave power source comprises a co-axial cable for feeding microwave power to the antenna.

In a third embodiment, the microwave power source and the microwave delivery device are essentially integrated and comprise an external focused microwave source for heating of varicose veins that does not require an intracorporeal antenna. The superposition of microwave energy could be controlled transcutaneously to heat only the vessel walls desired. This microwave heating method is completely external and requires no invasiveness.

For transcutaneous heating, the microwave source could be attached to or used in conjunction with an ultrasound probe or other imaging devices or systems. With this method, the ultrasound probe could be used to localize the targeted vein in real-time. The vein could be compressed in any suitable manner to temporarily stop blood flow, and then sealed closed with focused microwave heating. Doppler ultrasound could then be used to confirm that the vein has no flow. Such a method could be used with or without an intracorporeal antenna.

With any of the embodiments described herein, a Mylar balloon (or an inflatable balloon or device of other conductive material) could be placed on the end of a catheter that is inserted into the vein. The balloon could be partially inflated to ensure that the catheter stays in contact with the vein wall to assure uniform energy delivery.

The vascular ablation system preferably may include a built-in cooling system to reduce skin burns when the microwave power source is external and placed on the skin. The cooling system may be separate or integrated into the microwave power source, such as a system of cooling channels, which may also be integrated into the ultrasound probe or other imaging device. The system can also provide for temperature monitoring at the skin surface.

The vascular ablation system preferably may include a temperature monitoring, feedback and control system used with any of the embodiments described herein. Temperature monitoring may be accomplished via a thermosensor in the catheter, and/or an external non-invasive temperature monitoring device.

The vascular ablation system may also include a method of compression, such as ultrasound guided compression or any other suitable compressing of the vessel, to stop blood flow and co-apt the vein walls during microwave ablation using any of the embodiments and methods described herein.

Accordingly, it is one of the objects of the present disclosure to provide a method and system for the controlled delivery of microwave power to a vessel wall such as a vein.

It is a further object of the present invention to provide a method and device for the delivery of microwave power to treat vascular pathologies such as varicose veins.

It is another object of the present invention to provide a method and system for vascular ablation.

Numerous other advantages and features of the disclosure will become readily apparent from the following detailed description, from the claims and from the accompanying drawings in which like numerals are employed to designate like parts throughout the same.

### BRIEF DESCRIPTION OF THE DRAWINGS

A fuller understanding of the foregoing may be had by reference to the accompanying drawings wherein:

FIG. 1 is a schematic cross-sectional view of a first embodiment of the present invention, showing the antenna and microwave source relative to a vessel.

FIG. 2 is a schematic cross-sectional view of a second embodiment of the present invention, showing a radiating microwave antenna placed inside the vessel.

FIG. 3 is a schematic cross-sectional view of a third embodiment of the present invention, showing an integrated external microwave source and delivery device focused on an area inside the vessel.

FIG. 4 is a schematic cross-sectional view of an alternate embodiment of the present invention, showing a balloon used to maintain the position of an antenna relative to the vessel walls.

### DESCRIPTION OF DISCLOSED EMBODIMENT(S)

While the invention is susceptible of embodiment in many different forms, there is shown in the drawings and will be described herein in detail one or more embodiments of the present disclosure. It should be understood, however, that the present disclosure is to be considered an exemplification of the principles of the invention, and the embodiment(s) illustrated is/are not intended to limit the spirit and scope of the invention and/or the claims herein.

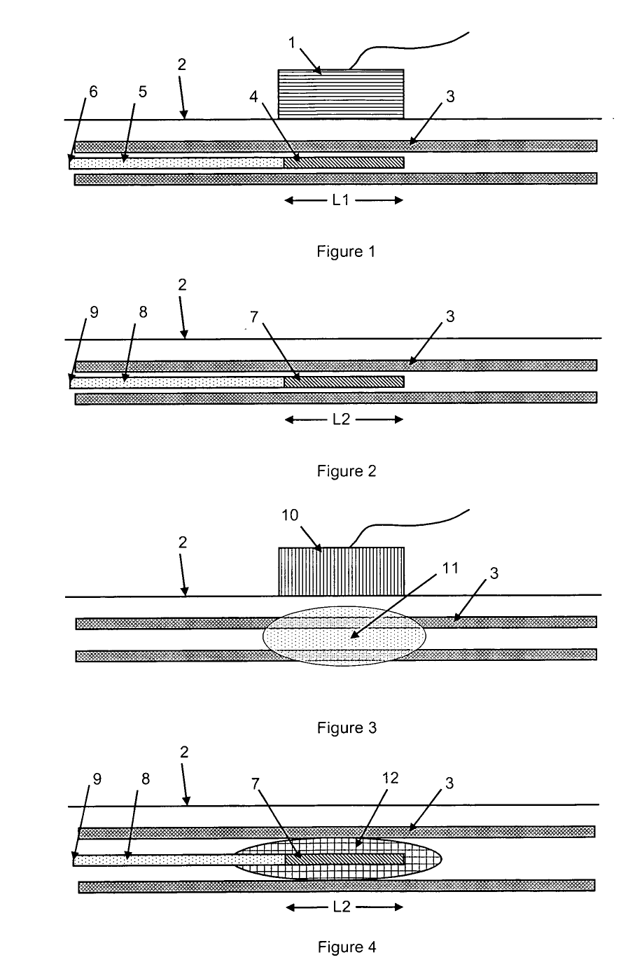

FIGS. 1-3 illustrate several embodiments of the vascular ablation method and system of the present disclosure is shown.

As illustrated in FIG. 1, a first embodiment of the present disclosure comprises a thin metallic wire antenna 4 positioned inside the vessel 3 by a non-radiating catheter 5. The antenna 4 may be purely metallic or contain a core or sections of ferrite or similar material to enhance the heating effect. For small, tortuous veins, the antenna/catheter should be flexible enough to migrate therethrough. An external microwave source 1 positioned proximate the skin surface 2 directs energy at the wire antenna 4 causing the antenna 4 to radiate locally, thereby focusing the microwave energy on the wall of the vessel 3 to heat and ablate the vessel 3. The length L1 of the antenna 4 is arbitrary. The placement catheter 5 is located at the proximal end 6.

As illustrated in FIG. 2, a second embodiment of the present disclosure comprises a coaxial cable 9 which feeds the radiating antenna 7 directly with microwave energy. That energy is radiated by the antenna 7 to the wall of the vessel 3. The antenna length L2 is fixed by the frequency of the microwave energy applied.

As illustrated in FIG. 3, a third embodiment of the present disclosure comprises an external microwave source 10 controlled in such a way as to focus radiated energy in a small volume 11 onto the vessel 3. The energy is applied transcutaneously.

In any of the three embodiments described above, a device such as a balloon may be used to assist in providing generally uniform energy delivery in the vessel. As illustrated in FIG. 4, the balloon 12, comprised of conductive material such as Mylar, is shown in use in the vessel 3 to hold the position of the antenna 7 relative to the vessel wall.

Further, the vascular method and system of the present disclosure may include the use of an ultrasound probe or other imaging system or device to guide the antennas into place in the vessels. The ultrasound probe may also house the microwave source, such as the external microwave source 1 shown in FIG. 1, or external microwave source 10 shown in FIG. 3. The ultrasound probe and/or the external microwave source 1 or 10, may also house a cooling system to be placed on the skin 2 to cool the skin. The ultrasound probe may also be used to compress the skin 2 and vessel 3 during use of any energy delivery system to stop blood flow and allow full treatment of the vessel wall. It should be understood that the vessel may be compressed in any suitable manner, and the use of the ultrasound probe is just one example of such compression.

Still further, a thermosensor or external thermometry system may be used to measure the temperature of the vessel wall and/or the skin surface and provide feedback. Temperature information may be used in a feedback loop to control the microwave power applied, location of focused heating, antenna placement or treatment duration.

It is to be understood that the embodiment(s) herein described is/are merely illustrative of the principles of the present invention. Various modifications may be made by those skilled in the art without departing from the spirit or scope of the claims which follow. For example, the antenna/catheter may include an LED or other indicator that can be observed through the skin or otherwise used to monitor position of the antenna, especially near a patient's saphenofemoral junction. Further, the antenna can be coated with any suitable material or coating to prevent the antenna from adhering to the clot forming in the vein and/or to the vein wall during use.

With respect to the delivery of energy to the vein, the embodiments disclosed herein may include both pulsed and continuous energy delivery. A foot pedal or any other suitable switch or trigger device may be incorporated to allow the user to selectively switch energy delivery on/off. Microwave ablation of veins may be achieved using continuous power application, or by sequentially treating segments of the vein and pulling the antenna back between each. Different power schedules/powers for large (e.g. >5 mm) and small veins can be used or delivered. Also, multiple external power sources with destructive/constructive interference capability may be incorporated and used in the disclosed embodiments. Any combination of external power sources are contemplated, not just microwave, but also, for example, high-frequency ultrasound (hiFU), radio frequency (RF), and any other suitable external power sources. Further, compression of the vessel can be used with any external power source(s) or combinations thereof.

Additionally, the embodiments disclosed herein may be used in combination with any imaging monitoring (CT, US, MRI, fluoroscopy, mammography, nuclear medicine, etc.). With respect to the use of ultrasound, the antenna/catheter may have an echogenic coating or surface for better US visualization. Feedback systems (temperature, doppler, reflected power, etc.) and audio or visual indicators may be incorporated and used to advise the user or operator to hold/change the current position or retraction rate. The disclosed embodiments can incorporate and use software for targeting (in combination with imaging guidance), similar to a biopsy guide with ultrasound. This could assure that all of the power sources are focused on the same target.

What is claimed is:

1. A device for delivery of ablative power to a vessel, comprising:
a wire antenna; and
an external power source.

2. The device of claim 1, wherein the external power source is a microwave power source.

3. The device of claim 1, further comprising an ultrasound probe mounted in conjunction with the power source.

4. The device of claim 1, further comprising a cooling device for cooling skin surface proximate the power source.

5. The device of claim 4, wherein the cooling device comprises a system of cooling channels located on the external power source.

6. The device of claim 4, further comprising a temperature monitor at the skin surface.

7. A device for delivery of ablative power to a vessel, comprising:
a thin, intralumenal antenna;
wherein the antenna is operatively connected to a power source.

8. The device of claim 7, wherein the power source is a microwave power source.

9. The device of claim 7, further comprising a means for maintaining relative positioning between the antenna and a wall of the vessel.

10. The device of claim 9, wherein the means for maintaining is a balloon of conductive material mounted on an antenna catheter.

11. The device of claim 10, wherein the conductive material is Mylar.

12. A device for delivery of microwave power to a vessel, comprising:
a focused, external microwave source.

13. The device of claim 12, further comprising an ultrasound probe mounted in conjunction with the microwave source.

14. A method for ablation of a vessel, comprising the steps of:
placing an image guidance device on a skin surface proximate the vessel;
applying pressure to the image guidance device sufficient to compress and close the vessel; and
delivering ablative power to the vessel.

15. The method of claim 14, wherein the ablative power is microwave power.

16. The method of claim 14, wherein the image guidance device is an ultrasound probe.
